(19) United States
(12) Patent Application Publication
(10) Pub. No.: US 2025/0319108 A1
(43) Pub. Date: Oct. 16, 2025

(54) COMBINATIONS

(71) Applicant: AN2 Therapeutics, Inc., Menlo Park, CA (US)

(72) Inventors: Michael Richard Kevin ALLEY, Santa Clara, CA (US); Eric Edward EASOM, Atherton, CA (US); Paul Brian ECKBURG, Mountain View, CA (US); Kevin Michael KRAUSE, San Francisco, CA (US); George H. TALBOT, Bradenton, FL (US)

(73) Assignee: AN2 Therapeutics, Inc., Menlo Park, CA (US)

(21) Appl. No.: 18/716,814

(22) PCT Filed: Dec. 6, 2022
(86) PCT No.: PCT/US2022/080986

§ 371 Date: Jun. 5, 2024

Related U.S. Application Data
(60) Provisional application No. 63/286,989, filed on Dec. 7, 2021.
(60) Provisional application No. 63/416,059, filed on Oct. 14, 2022.

(51) Int. Cl. A61K 31/69; A61K 31/133; A61K 31/437; A61K 31/438; A61K 31/496; A61K 31/7048; A61K 31/7052; A61P 31/04

(52) U.S. Cl. CPC A61K 31/69; A61K 31/133; A61K 31/437; A61K 31/438; A61K 31/496; A61K 31/7048; A61K 31/7052; A61P 31/04

(57) ABSTRACT

This invention relates to, among other items, treating non-tuberculosis Mycobacleria-associated disease in a human with epetraborole.

### PRIORITY CLAIM

This application claims benefit to U.S. Provisional Application Ser. No. 63/286,989, filed Dec. 7, 2021, and 63/416,059, filed Oct. 14, 2022, each of which are incorporated herein by reference in their entirety.

### BACKGROUND OF THE INVENTION

Non-tuberculous mycobacteria (NTM) infections are increasing in humans. Standard of care calls for 18-24 months of treatment with a minimum of three antibiotics. Despite this, treatment outcomes remain poor.

New methods of treatment for NTM infections would represent an advancement in the art.

### SUMMARY OF THE INVENTION

In a first aspect, the invention provides a method of treating a non-tuberculosis Mycobacteria infection in a human, comprising: administering epetraborole or a hydrate, solvate, or salt thereof, and ethambutol to the human, thereby treating the non-tuberculosis Mycobacteria infection in the human.

In a second aspect, the invention provides a method of treating a non-tuberculosis Mycobacteria-associated disease in a human, comprising: administering epetraborole or a hydrate, solvate, or pharmaceutically acceptable salt thereof, and ethambutol to the human, thereby treating the non-tuberculosis Mycobacteria-associated disease in the human.

### DETAILED DESCRIPTION OF THE INVENTION

### I. Definitions and Abbreviations

In order that the application may be more completely understood, several definitions are set forth below. Such definitions are meant to encompass grammatical equivalents.

The term “about” in relation to a reference numerical value can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

Specific embodiments disclosed herein can be further limited in the claims using “consisting of” or “consisting essentially of” language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

The terms “a,” “an,” “the” and similar referents used in the context of describing the disclosure (especially in the context of the following claims) are to be construed to cover both the singular and the plural, unless otherwise indicated herein or clearly contradicted by context.

The abbreviations used herein generally have their conventional meaning within the chemical and biological arts.

The following abbreviations have been used: AMK-Amikacin; ATCC-American Type Culture Collection; CA-MHB-Cation-adjusted Mueller Hinton broth; CFU-Colony forming unit; CLSI-Clinical and Laboratories Standards Institute; CLR-Clarithromycin; EBO-Epetraborole hydrochloride; EMB-Ethambutol; IC-Inhibitory Concentration; LeuRS-Leucyl-tRNA synthetase; MAC-Mycobacterium avium complex; MIC-Minimum inhibitory concentration; NTM-Nontuberculous mycobacteria; OADC-Oleic acid, bovine albumin, dextrose and catalase; RFB-Rifabutin; spp.-Species; subspp.- Subspecies.

“Epetraborole of the invention,” as used herein refers to epetraborole, salts (e.g. pharmaceutically acceptable salts), solvates and hydrates of these compounds. “Moiety” refers to a radical of a molecule that is attached to the remainder of the molecule.

The symbol whether utilized as a bond or displayed perpendicular to a bond, indicates the point at which the displayed moiety is attached to the remainder of the molecule.

The term “pharmaceutically acceptable salt” is meant to include a salt of an epetraborole of the invention which is prepared with relatively nontoxic acids or bases, depending on the particular substituents found on the compounds described herein. When epetraborole of the invention contain relatively acidic functionalities, base addition salts can be obtained by contacting the neutral form of such compounds with a sufficient amount of the desired base, either neat or in a suitable inert solvent.

Examples of pharmaceutically acceptable base addition salts include sodium, potassium, calcium, ammonium, organic amino (such as choline or diethylamine or amino acids such as d-arginine, 1-arginine, d-lysine or 1-lysine), or magnesium salt, or a similar salt. When epetraborole of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science 66: 1-19 (1977)). Certain specific compounds of the invention contain both basic and acidic functionalities that allow the compounds to be converted into either base or acid addition salts.

The neutral forms of the compounds are preferably regenerated by contacting the salt with a base or acid and isolating the parent compounds in the conventional manner. The parent form of the compound differs from the various salt forms in certain physical properties, such as solubility in polar solvents.

In addition to salt forms, the invention provides compounds which are in a prodrug form. Prodrugs of the compounds described herein readily undergo chemical changes under physiological conditions to provide the compounds of the invention. Additionally, prodrugs can be converted to the compounds of the invention by chemical or biochemical methods in an ex vivo environment.

Certain compounds of the invention can exist in unsolvated forms as well as solvated forms, including hydrated forms. In general, the solvated forms are equivalent to unsolvated forms and are encompassed within the scope of the invention. Certain compounds of the invention may exist in multiple crystalline or amorphous forms.

Certain compounds of the invention possess asymmetric carbon atoms (optical centers) or double bonds; the racemates, diastereomers, geometric isomers and individual isomers are encompassed within the scope of the invention. The graphic representations of racemic, ambiscalemic and scalemic or enantiomerically pure compounds used herein are taken from Maehr, J. Chem. Ed. 1985, 62: 114-120. Solid and broken wedges are used to denote the absolute configuration of a stereocenter unless otherwise noted. When the compounds described herein contain olefinic double bonds or other centers of geometric asymmetry, and unless specified otherwise, it is intended that the compounds include both E and Z geometric isomers.

Likewise, all tautomeric forms are included.

Compounds of the invention can exist in particular geometric or stereoisomeric forms. The invention contemplates all such compounds, including cis- and trans-isomers, (−)- and (+)-enantiomers, (R)- and (S)-enantiomers, diastereomers, (D)-isomers, (L)-isomers, the racemic mixtures thereof, and other mixtures thereof, such as enantiomerically or diastereomerically enriched mixtures, as falling within the scope of the invention. Additional asymmetric carbon atoms can be present in a substituent such as an alkyl group. All such isomers, as well as mixtures thereof, are intended to be included in this invention.

Optically active (R)- and (S)-isomers and d and l isomers can be prepared using chiral synthons or chiral reagents, or resolved using conventional techniques. If, for instance, a particular enantiomer of a compound of the invention is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

The compounds of the invention may also contain unnatural proportions of atomic isotopes at one or more of the atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds of the invention, whether radioactive or not, are intended to be encompassed within the scope of the invention.

The term “pharmaceutically acceptable carrier” or “pharmaceutically acceptable vehicle” refers to any formulation or carrier medium that provides the appropriate delivery of an effective amount of an active agent as defined herein, does not interfere with the effectiveness of the biological activity of the active agent, and that is sufficiently non-toxic to the host or patient. Representative carriers include water, oils, both vegetable and mineral, cream bases, lotion bases, ointment bases and the like. These bases include suspending agents, thickeners, penetration enhancers, and the like. Their formulation is well known to those in the art of cosmetics and topical pharmaceuticals. Additional information concerning carriers can be found in Remington: The Science and Practice of Pharmacy, 21st Ed., Lippincott, Williams & Wilkins (2005) which is incorporated herein by reference.

The term “excipients” is conventionally known to mean carriers, diluents and/or vehicles used in formulating drug compositions effective for the desired use.

The term “microbial infection” or “infection by a microorganism” refers to any infection of a host tissue by an infectious agent including, but not limited to, bacteria or protozoa (see, e.g., Harrison's Principles of Internal Medicine, pp. 93-98 (Wilson et al., eds., 12th ed. 1991); Williams et al., J. of Medicinal Chem. 42:1481-1485 (1999), herein each incorporated by reference in their entirety).

“Biological medium,” as used herein refers to both in vitro and in vivo biological milieus. Exemplary in vitro “biological media” include, but are not limited to, cell culture, tissue culture, homogenates, plasma and blood. In vivo applications are generally performed in mammals, preferably humans.

“Inhibiting” and “blocking,” are used interchangeably herein to refer to the partial or full blockade of enzyme. In an exemplary embodiment, the enzyme is a tRNA synthetase.

Boron is able to form additional covalent or dative bonds with oxygen, sulfur or nitrogen under some circumstances in this invention.

Embodiments of the invention also encompass compounds that are poly- or multi-valent species, including, for example, species such as dimers, trimers, tetramers and higher homologs of the compounds of use in the invention or reactive analogues thereof.

“Salt counterion”, as used herein, refers to positively charged ions that associate with a compound of the invention when the boron is fully negatively or partially negatively charged. Examples of salt counterions include H+, H₃O+, ammonium, potassium, calcium, magnesium (such as choline or diethylamine or amino acids such as d-arginine, 1-arginine, d-lysine or 1-lysine) and sodium.

The compounds comprising a boron bonded to a carbon and three heteroatoms (such as three oxygens described in this section) can optionally contain a fully negatively charged boron or partially negatively charged boron. Due to the negative charge, a positively charged counterion may associate with this compound, thus forming a salt. Examples of salt counterions include H+, H₃O+, ammonium, potassium, calcium, magnesium (such as choline or diethylamine or amino acids such as d-arginine, 1-arginine, d-lysine or 1-lysine) and sodium. The salts of the compounds are implicitly contained in descriptions of these compounds.

A ‘treatment-refractory’ infection, as used herein, refers to an infection in a human in which the sputum culture remains positive for the non-tuberculosis Mycobacteria after 6 months of treatment with azithromycin (or clarithromycin), rifampicin (or rifabutin), and ethambutol.

### II. Introduction

It has been found that epetraborole is useful in the treatment of certain non-tuberculosis Mycobacteria infections. It has additionally been found that combinations of epetraborole and ethambutol are useful in the treatment of certain non-tuberculosis Mycobacteria infections.

### III. Compounds

### Epetraborole, or a Salt, Hydrate, or Solvate Thereof

Epetraborole has a structure according to the following formula:

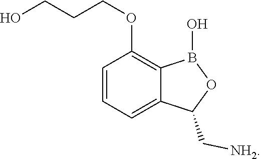

Epetraborole can be produced according the methods such as those disclosed in PCT Pat Pub WO 2008/157726 (PCT Pat App PCT/US2008/07550); U.S. Pat. No. 7,816,344 (U.S. patent application Ser. No. 12/142,692); PCT Pat Pub WO 2011/127143 (PCT Pat App PCT/US2011/031384); and U.S. Pat. No. 9,243,003 (U.S. patent application Ser. No. 13/639,594).

The epetraborole can form a hydrate with water, solvates with alcohols such as methanol, ethanol, propanol, and the like; adducts with amino compounds, such as ammonia, methylamine, ethylamine, and the like; adducts with acids, such as formic acid, acetic acid and the like; complexes with ethanolamine, quinoline, amino acids, and the like.

In an exemplary embodiment, the invention provides epetraborole, or a salt, hydrate or solvate thereof, or a combination thereof. In an exemplary embodiment, the invention provides epetraborole, or a salt, hydrate or solvate thereof. In an exemplary embodiment, the invention provides epetraborole, or a salt thereof. In an exemplary embodiment, the salt is a pharmaceutically acceptable salt. In an exemplary embodiment, the invention provides a hydrochloride salt of epetraborole. In an exemplary embodiment, the invention provides epetraborole, or a hydrate thereof. In an exemplary embodiment, the invention provides epetraborole, or a solvate thereof.

### Ntm Infection Standard of Treatment

According to published recommendations, NTM infections in humans can be treated through a combination of ethambutol, rifampin or rifabutin, and a macrolide (clarithromycin or azithromycin).

### Ethambutol, or a Salt, Hydrate, or Solvate Thereof

Ethambutol has a structure according to the following formula:

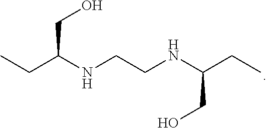

Ethambutol is commercially produced by a number of manufactuers, such as Sanofi, Cadila, Lupin, and Delmar.

### Rifampin, or a Salt, Hydrate, or Solvate Thereof

Rifampin, also known as rifampicin, is commercially produced by a number of manufactuers, such as Novartis, Otto Brandes, Arudavis Labs, and Sichuan Long March Pharma.

### Rifabutin, or a Salt, Hydrate, or Solvate Thereof

Rifabutin is commercially produced by a number of manufactuers, such as Pfizer, Chongqing Huapont Pharma, Lupin, and Guangzhou Tosun Pharma.

### Macrolide (Clarithromycin or Azithromycin)

Clarithromycin is commercially produced by a number of manufactuers, such as Sandoz, Century Pharmaceuticals, Teva, Wockhardt, and Alembic.

Azithromycin is commercially produced by a number of manufactuers, such as Pfizer, Sandoz, Teva, Alembic, and Lupin.

Synergistic Effects with Epetraborole and Ethambutol

Synergistic effects between epetraborole and ethambutol have been discovered which can result in more effective treatments of NTM infections.

### IV. Methods

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, to the human, thereby treating the non-tuberculosis Mycobacteria infection in the human. In an exemplary embodiment, the method further comprises administering a rifamycin, or a hydrate, solvate, or salt thereof, to the human. In an exemplary embodiment, the rifamycin is rifampicin (rifampin), or a hydrate, solvate, or salt thereof. In an exemplary embodiment, the rifamycin is rifabutin, or a hydrate, solvate, or salt thereof. In an exemplary embodiment, the rifamycin is rifapentine, or a hydrate, solvate, or salt thereof. In an exemplary embodiment, the rifamycin is rifaximin, or a hydrate, solvate, or salt thereof. In an exemplary embodiment, the method further comprises administering a macrolide, or a hydrate, solvate, or salt thereof, to the human. In an exemplary embodiment, the macrolide is clarithromycin or a hydrate, solvate, or salt thereof. In an exemplary embodiment, the macrolide is azithromycin or a hydrate, solvate, or salt thereof. In an exemplary embodiment, the epetraborole is a salt of the epetraborole, and the salt is a pharmaceutically acceptable salt. In an exemplary embodiment, the epetraborole is epetraborole hydrochloride. In an exemplary embodiment, the non-tuberculosis Mycobacteria is M. intracellulare or M. avium. In an exemplary embodiment, for any of the methods in this paragraph, rifamycin, or a hydrate, solvate, or salt thereof, is not administered to the human. In an exemplary embodiment, for any of the methods in this paragraph, rifabutin, or a hydrate, solvate, or salt thereof, is not administered to the human. In an exemplary embodiment, for any of the methods in this paragraph, a macrolide, or a hydrate, solvate, or salt thereof, is not administered to the human. In an exemplary embodiment, for any of the methods in this paragraph, azithromycin, or a hydrate, solvate, or salt thereof, is not administered to the human. In an exemplary embodiment, for any of the methods in this paragraph, clarithromycin, or a hydrate, solvate, or salt thereof, is not administered to the human.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and a rifamycin, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and rifabutin, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and a macrolide, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and clarithromycin, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and azithromycin, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and a rifamycin, or a hydrate, solvate, or salt thereof, and a macrolide, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and a rifamycin, or a hydrate, solvate, or salt thereof, and clarithromycin, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and a rifamycin, or a hydrate, solvate, or salt thereof, and azithromycin, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and rifabutin, or a hydrate, solvate, or salt thereof, and a macrolide, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and rifabutin, or a hydrate, solvate, or salt thereof, and clarithromycin, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and rifabutin, or a hydrate, solvate, or salt thereof, and azithromycin, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In an exemplary embodiment, the invention provides a method of treating a non-tuberculosis Mycobacteria infection in a human, comprising: administering epetraborole or a hydrate, solvate, or salt thereof, and a rifamycin, or a hydrate, solvate, or salt thereof, to the human, thereby treating the non-tuberculosis Mycobacteria infection in the human. In an exemplary embodiment, the invention provides a method of treating a non-tuberculosis Mycobacteria infection in a human, comprising: administering epetraborole or a hydrate, solvate, or salt thereof, and rifabutin, or a hydrate, solvate, or salt thereof, to the human, thereby treating the non-tuberculosis Mycobacteria infection in the human. In an exemplary embodiment, the invention provides a method of treating a non-tuberculosis Mycobacteria infection in a human, comprising: administering epetraborole or a hydrate, solvate, or salt thereof, and a macrolide, or a hydrate, solvate, or salt thereof, to the human, thereby treating the non-tuberculosis Mycobacteria infection in the human. In an exemplary embodiment, the invention provides a method of treating a non-tuberculosis Mycobacteria infection in a human, comprising: administering epetraborole or a hydrate, solvate, or salt thereof, and clarithromycin, or a hydrate, solvate, or salt thereof, to the human, thereby treating the non-tuberculosis Mycobacteria infection in the human. In an exemplary embodiment, the invention provides a method of treating a non-tuberculosis Mycobacteria infection in a human, comprising: administering epetraborole or a hydrate, solvate, or salt thereof, and azithromycin, or a hydrate, solvate, or salt thereof, to the human, thereby treating the non-tuberculosis Mycobacteria infection in the human. In an exemplary embodiment, the invention provides a method of treating a non-tuberculosis Mycobacteria infection in a human, comprising: administering epetraborole or a hydrate, solvate, or salt thereof, and a rifamycin, or a hydrate, solvate, or salt thereof, and a macrolide, or a hydrate, solvate, or salt thereof, to the human, thereby treating the non-tuberculosis Mycobacteria infection in the human. In an exemplary embodiment, the invention provides a method of treating a non-tuberculosis Mycobacteria infection in a human, comprising: administering epetraborole or a hydrate, solvate, or salt thereof, and ethambutol, or a hydrate, solvate, or salt thereof, and a rifamycin, or a hydrate, solvate, or salt thereof, and clarithromycin, or a hydrate, solvate, or salt thereof, to the human, thereby treating the non-tuberculosis Mycobacteria infection in the human. In an exemplary embodiment, the invention provides a method of treating a non-tuberculosis Mycobacteria infection in a human, comprising: administering epetraborole or a hydrate, solvate, or salt thereof, and ethambutol, or a hydrate, solvate, or salt thereof, and a rifamycin, or a hydrate, solvate, or salt thereof, and azithromycin, or a hydrate, solvate, or salt thereof, to the human, thereby treating the non-tuberculosis Mycobacteria infection in the human. In an exemplary embodiment, the invention provides a method of treating a non-tuberculosis Mycobacteria infection in a human, comprising: administering epetraborole or a hydrate, solvate, or salt thereof, and rifabutin, or a hydrate, solvate, or salt thereof, and a macrolide, or a hydrate, solvate, or salt thereof, to the human, thereby treating the non-tuberculosis Mycobacteria infection in the human. In an exemplary embodiment, the invention provides a method of treating a non-tuberculosis Mycobacteria infection in a human, comprising: administering epetraborole or a hydrate, solvate, or salt thereof, and clarithromycin, or a hydrate, solvate, or salt thereof, to the human, thereby treating the non-tuberculosis Mycobacteria infection in the human. In an exemplary embodiment, the invention provides a method of treating a non-tuberculosis Mycobacteria infection in a human, comprising: administering epetraborole or a hydrate, solvate, or salt thereof, and azithromycin, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium. In an exemplary embodiment, for any of the methods in this paragraph, ethambutol, or a hydrate, solvate, or salt thereof, is not administered to the human.

In another aspect, the invention provides a method of treating a non-tuberculosis Mycobacteria infection in a human, comprising: administering epetraborole or a hydrate, solvate, or salt thereof, and ethambutol, or a hydrate, solvate, or salt thereof, and a macrolide, or a hydrate, solvate, or salt thereof, to the human, thereby treating the non-tuberculosis Mycobacteria infection in the human. In an exemplary embodiment, the non-tuberculosis Mycobacteria is M. intracellulare or M. avium.

In an exemplary embodiment, the non-tuberculosis Mycobacteria is rapidly growing. In an exemplary embodiment, the non-tuberculosis Mycobacteria is rapidly growing, and is M. abscessus, M. arabiense, M. aromaticivorans, M. bacteremicum, M. barrassiae, M. bourgelatii, M. celeriflavum, M. chelonae, M. crocinum, M. fukienense, M. hippocampi, M. insubricum, M. iranicum, M. litorale, M. llatzerense, M. monacense, M. pallens, M. rufum, M. rutilum, M. salmoniphilum, M. sediminis, or Mycobacterium setense, or a combination thereof.

In an exemplary embodiment, the non-tuberculosis Mycobacteria is slowly growing. In an exemplary embodiment, the non-tuberculosis Mycobacteria is slowly growing, and is M. algericum, M. alsiense, M. arosiense, M. bouchedurhonense, M. engbaekii, M. europaeum, M. fragae, M. heraklionense, M. indicus pranii, M. koreense, M. kumamotonense, M. kyorinense, M. lepromatosis, M. liflandii, M. longobardum, M. mantenii, M. marseillense, M. minnesotense, M. noviomagense, M. paraffinicum, M. paragordonae, M. parakoreense, M. paraseoulense, M. paraterrae, M. riyadhense, M. senuense, M. seoulense, M. sherrisii, M. shigaense, M. shinjukuense, M. simulans, M. sinense, M. stomatepiae, M. timonense, M. vulneris, or M. yongonense, or a combination thereof.

In an exemplary embodiment, the non-tuberculosis Mycobacteria is selected from the group consisting of M. abscessus, M. avium complex (MAC), M. chelonae, M. fortuitum, M. gordonae, M. kansasii, M. mucogenicum, M. peregrinum, and M. xenopi. In an exemplary embodiment, the non-tuberculosis Mycobacteria is selected from the group consisting ofM. abscessus, M. avium complex (MAC), M. fortuitum complex, M. gordonae, M. kansasii, and M. xenopi.

In an exemplary embodiment, the non-tuberculosis Mycobacteria is Mycobacterium avium complex. In an exemplary embodiment, the non-tuberculosis Mycobacteria is M. avium, M. intracellulare, M. narseillaise, H. timonense, M. bouchedurhonense, M. colombiense, M. vulneris. or M. chimaera. In an exemplary embodiment, the non-tuberculosis Mycobacteria is M. intracellulare. In an exemplary embodiment, the non-tuberculosis Mycobacteria is M. intracellulare subsp. intracellulare or M. intracellulare subsp.. In an exemplary embodiment, the non-tuberculosis Mycobacteria is M. avium.

In an exemplary embodiment, the human further has a disease which is cystic fibrosis, chronic obstructive pulmonary disease, or chronic thromboembolic pulmonary hypertension. In an exemplary embodiment, the human further has a disease which is an interstitial lung disease, post-inflamnmatory lung fibrosis, bronchiectasis, a neoplastic disease, diabetes mellitus, bronchial asthma, hypothyreosis, mediastinal cyst, or rheumatoid arthritis. In an exemplary embodiment, the human further has a disease which is interstitial lung disease, and the interstitial lung disease is idiopathic pulmonary fibrosis, sarcoidosis, or proteinosis.

In an exemplary embodiment, the human further has a neoplastic disease, and the neoplastic disease is myelofibrosis or lung cancer. In an exemplary embodiment, the human previously suffered from tuberculosis.

In an exemplary embodiment, the infection is in the lung of the human. In an exemplary embodiment, the infection is in two or more organs in the body. In an exemplary embodiment, the infection is in the lymph nodes.

In an exemplary embodiment, the infection is treatment-naive. In an exemplary embodiment, the infection is treatment-refractory.

In a further aspect, the invention provides a method of treating a non-tuberculosis Mycobacteria-associated disease in a human, comprising: administering epetraborole or a hydrate, solvate, or pharmaceutically acceptable salt thereof, and ethambutol to the human, thereby treating the non-tuberculosis Mycobacteria-associated disease in the human. In an exemplary embodiment, the method further comprises administering rifabutin, or a salt thereof, or rifamycin, or a salt thereof, to the human. In an exemplary embodiment, the method further comprises administering a macrolide, or a salt thereof, to the human. In an exemplary embodiment, the macrolide is clarithromycin or azithromycin. In an exemplary embodiment, the epetraborole is a salt of the epetraborole, and the salt is a pharmaceutically acceptable salt. In an exemplary embodiment, the epetraborole is epetraborole hydrochloride. In an exemplary embodiment, the non-tuberculosis Mycobacteria-associated disease is non-tuberculosis Mycobacteria-pulmonary disease, disseminated non-tuberculosis Mycobacteria disease, or non-tuberculosis Mycobacteria-associated lymphadenitis. In an exemplary embodiment, the non-tuberculosis Mycobacteria-associated disease is Mycobacterium avium complex (MAC) pulmonary disease, disseminated Mycobacterium avium complex (MAC) disease, and Mycobacterium avium complex (MAC)-associated lymphadenitis. In an exemplary embodiment, the non-tuberculosis Mycobacteria-associated disease is nodular bronchiectasis. In an exemplary embodiment, the non-tuberculosis Mycobacteria-associated disease is fibrocavitary.

In an exemplary embodiment, the non-tuberculosis Mycobacteria-associated disease is treatment-naive. In an exemplary embodiment, the non-tuberculosis Mycobacteria-associated disease is treatment-refractory.

In an exemplary embodiment, the disease is treated through oral administration of a compound of the invention. In an exemplary embodiment, the disease is treated through intravenous administration of a compound of the invention.

In an exemplary embodiment, the disease is treated through subcutaneous administration of a compound of the invention.

### V. Pharmaceutical Formulation

In another aspect, the invention provides a pharmaceutical formulation comprising: a) epetraborole or a salt, hydrate, or solvate thereof; and b) a pharmaceutically acceptable excipient.

It is to be understood that the present invention covers all combinations of aspects and/or embodiments, as well as suitable, convenient and preferred groups described herein.

The invention is further illustrated by the Examples that follow. The Examples are not intended to define or limit the scope of the invention.

### EXAMPLES

### Example 1

Minimum inhibitory concentration (MIC) determinations: MIC values for the putative EBO resistant mutants were determined by broth microdilution method (BMD) in cation-adjusted Mueller Hinton broth according to Clinical and Laboratory Standards Institute document M24-A3 (Clinical and Laboratory Standards Institute. Susceptibility Testing of Mycobacteria, Nocardia spp., and Other Aerobic Actinomycetes. 3rd ed. CLSI standard M24. Clinical and Laboratory Standards Institute, Wayne, PA: 2018). Agar MIC values were determined as essentially described by CLSI M24-A3 using 7H10 Middlebrook agar and 5% OADC.

The in vitro activity of EBO, clarithromycin (CLR), ethambutol (EMB), rifabutin, amikacin and bedaquiline was tested against 5 MAC strains and 2 rapidly-growing mycobacterial strains. MIC results are presented in the following table.

TABLE 1
In Vitro Activity (mg/L)

| | M. | | | | | M. | M. |
|---|---|---|---|---|---|---|---|
| | avium | M. | M. | M. | M. | abscessus | peregrinum |
| | ATCC | avium | chimaera | intracellulare | intracellulare | ATCC | ATCC |
| Drug | 700858 | 2285R | 20-S-05 | 20-S-13 | DNA000111 | 19977 | 700686 |
| EBO | 2 | 0.5 | 2 | 0.25 | 0.5 | 0.06 | 0.06 |
| CLR | 2 | 0.125 | 4 | >128 | 2 | 16 | 1 |
| RFB | 0.25 | 0.125 | 1 | 0.06 | 2 | 32 | 8 |
| EMB | 8 | 16 | 4 | 64 | 32 | 32 | 8 |
| AMK | 16 | 8 | 32 | 20 | 16 | 64 | 2 |
| BDQ | 0.06 | 0.03 | 0.06 | >0.5 | 0.5 | 0.5 | 0.06 |

### EXAMPLE 2

### Pairwise Combinations of Epetraborole

Epetraborole hydrochloride is small polar molecule with a novel mechanism of action (MoA) that has broad-spectrum antibacterial activity including activity against mycobacteria. The current standard of care for Mycobacterium avium complex (MAC) pulmonary disease involves a macrolide, ethambutol and a rifamycin. The activity of epetraborole hydrochloride was assessed in pairwise combinations with clarithromycin, rifabutin and ethambutol against 7 nontuberculous mycobacteria including 5 slowly growing nontuberculous mycobacteria and 2 rapidly growing mycobacteria. Epetraborole hydrochloride activity was also assessed in the presence of amikacin and bedaquiline. Results are shown below in Table 2.

### Materials and Methods

### Chemicals

EBO was obtained from AN2 Therapeutics Inc, bedaquiline was purchased from 1Click Chemistry (Kendall Park, NJ), clarithromycin was purchased from Carbosynth (San Diego, CA), amikacin, ethambutol and rifabutin were purchased from Sigma-Aldrich, (St. Louis, MO). Frozen stocks of the drugs were prepared at 5 or 2 mg/ml and frozen in aliquots at −20° C. On the day of the experiment the stocks were thawed and diluted to the appropriate concentration. Middlebrook 7H9 broth and agar, CA-MH agar and Middlebrook OADC were all purchased from Becton-Dickinson and Company (Sparks, MD).

### Strains

M. avium 2285R (Verma et al. Microbiol (2019) 10: 693) and M. intracellulare DNA000111 were obtained from Diane Ordway (Colorado State University, Fort Collins, CO). M. chimaera 20-S-05 and the macrolide/bedaquiline-resistant M. intracellulare 20-S-13 isolate were obtained from Barbara Brown-Elliott (Mycobacteria/Nocardia Research Laboratory, University of Texas Health Science Center, Tyler Tx). The strainsM. abscessus ATCC 19977, M. avium ATCC 700898 and M. peregrinum ATCC 700686 were obtained from the American Type Culture Collection (ATCC, Manassas, VA). Stocks of isolates were prepared and frozen at −80° C. Fresh cultures of each isolate (1-2 weeks) were grown on either 7H10 agar plus 5% OADC or CAMH agar plus 5% OADC for use in each experiment.

### Antibacterial Synergy Testing

The checkerboard methodology was used to test for synergy and antagonism using Middlebrook 7H9+5% OADC (Dubos et al. Am Rev Tuberc (1947) 56:334-45) as the microbiology growth media. The first antibiotic of the combination, EBO, was serially diluted along the x-axis, whereas the second antibiotic was diluted along the y- axis. Synergistic or antagonistic activity was determined using the sum of the fractional inhibitory concentration (EFIC) index. The FIC index is calculated as the sum of FIC A+FIC B, where FIC A is the MIC of drug A in the combination of drugs A and B divided by the MIC of drug A alone, plus the MIC of drug B in the combination of drugs A and B divided by the MIC of drug B alone. A combination of drugs is considered synergistic when the FIC is <0.5, additive when the FIC is >0.5 to 1, indifferent when the FIC is >1 to 2, and antagonistic when the FIC is >2 (EUCAST. Clin. Microbiol. Infect. (2000) 6: 503-508).

TABLE 2
Summary Activity of EBO Drug Combinations

| In | M. | | | | | M. | M. |
|---|---|---|---|---|---|---|---|
| Combination | avium | M. | M. | M. | M. | abscessus | peregrinum |
| with | ATCC | avium | chimaera | intracellulare | intracellulare | ATCC | ATCC |
| EBO | 700898 | 2285R | 20-S-05 | 20-S-13 | DNA000111 | 19977 | 700686 |
| CLR | I | A | I | NE | I | I | I |
| RFB | I | I | I | I | A | I | I |
| EMB | S | A | A | I | S | I | I |
| AMK | I | I | I | I | I | I | A |
| BDQ | A | I | I | NE | I | I | I |

AMK = Amikacin, BDQ = Bedaquiline, CLR = Clarithromycin, EBO = epetraborole hydrochloride, EMB = Ethambutol, RFB = Rifabutin, I = Indifferent, A = Additive, S = Synergistic. NE = No endpoint due to MIC of second drug being out of range, CLR MIC >128 mg/L, BDQ >0.5 mg/L.

### Results and Discussion

The in vitro activity of EBO was tested in the presence of key components of the standard of care drugs for the treatment of MAC pulmonary disease, clarithromycin, ethambutol, rifabutin as well as other known active NTM drugs, amikacin and bedaquiline. The activity of EBO was not antagonized by any of these drugs with any of the NTM strains we tested. In most cases, especially for the two rapidly growing NTM strains, M. abscessus ATCC 19977 and M. peregrinum ATCC 700686, EBO activity was indifferent to the addition of a second drug. The sole exception was ethambutol where synergy was observed with 2 strains and additivity with an additional 2 strains out of a total of 5 MAC strains tested. Interestingly, the clarithromycin resistant strain, M. intracellulare 20-S-13, was the only MAC strain that showed indifference between ethambutol and EBO and this strain had the highest ethambutol MIC with a value of 64 mg/L.

### CONCLUSIONS

No antagonisms were observed with any strain or EBO combination, and most interactions were largely indifferent especially for the two rapidly growing NTM strains, M. abscessus ATCC 19977 and M. peregrinum ATCC 700686. The sole exception was ethambutol where synergy was observed with 2 strains and additivity with an additional 2 strains out of a total of 5 MAC strains tested. Interestingly, the MAC strain with the highest ethambutol MIC value of 64 mg/L was the only strain that showed indifference.

### Example 3

### Spontaneous Resistance Frequency Determination:

The RF of M. avium ATCC 700898 at 2×, 4× and 8× the MIC (8 mg/L) of EBO was determined, as was the RF of EBO combined with CLR, RBT, AMK or EMB. MICs of selected EBO mutants were determined against AMK, BDQ, CLR, RBT, EMB, and clofazimine (CFZ) and the mutants were further characterized by genomic DNA analysis. Resistant colonies were confirmed by replica plating on agar plates containing antibiotic at the same concentration used to select resistance. Control plates containing no drug were prepared for inoculum determination. The RF was calculated by dividing the total CFU/mL of resistant colonies by the total CFU/mL of the inoculum.

The spontaneous resistance frequency for EBO ranged from 1.58×10⁻⁷ to 8.48×10⁻⁹ when selected on 2-8× agar MIC (Table 3). EBO resistance frequency was thus very similar to observed to that of standard-of-care (SOC) anitbacterials (Table 4). However, the addition of EMB, CLR, RFB, or AMK to EBO significantly lowered the resistance frequency more than 700-fold to both drugs (Table 4). Further characterization of the EBO resistant mutants showed that the MIC value for EBO increased 128-fold or greater, while the MIC values for amikacin, bedaquiline, clofazimine, clarithromycin and ethambutol did not change more than 4-fold (Table 5). The only drug tested that changed more than 4-fold from the wild-type MIC value was rifabutin but this was with a single EBO resistant mutant, 64-4A, which shifted 8-fold (Table 5). However, this was only with a single MIC value as its duplicate was only 4-fold different from wild-type. An 8-fold variance has been previously reported for this strain, M. avium ATCC 700898, with the related rifamycin, rifampin (Zelinski C, KillianSC, Sulivan N, Allen S. Mycobacterium avium ssp. avium ATCC 700898 (QC) culture differences demonstrate variable MIC susceptibility results in the Sensititre® SLOMYCO plate. ASM General Meeting 2010, San Diego CA C-153. This 8-fold difference is within error. Activity of the key antimycobacterials tested was not impacted by EBO resistance suggesting that cross-resistance did not occur.

TABLE 3
In Vitro Resistance Frequency of EBO in M. avium ATCC 700898

| | Agar MIC | Selection | Resistance |
|---|---|---|---|
| Drug | (mg/L) | Concentration (mg/L) | Frequency |
| EBO | 8 | 16 | 1.58 × 10⁻⁷ |
| | | 32 | 1.21 × 10⁻⁸ |
| | | 64 | 8.49 × 10⁻⁹ |

TABLE 4
In Vitro Resistance Frequency of SOC antibacterials alone
and with EBO (2 × MIC) in M. avium ATCC 700898

| | | | | +EBO |
|---|---|---|---|---|
| | | Selection | Monotherapy | (2 × MIC) |
| | Agar MIC | Concentration | Resistance | Resistance |
| Drug | (mg/L) | (mg/L) | Frequency | Frequency |
| CLR | 4 | 32 | 1.59 × 10⁻⁷ | <1.49 × 10⁻¹⁰ |
| RFB | 0.25 | 2 | 1.03 × 10⁻⁸ | <1.49 × 10⁻¹⁰ |
| AMK | 32 | 128 | 1.21 × 10⁻⁸ | <1.49 × 10⁻¹⁰ |
| EMB | 8 | 24 | 3.13 × 10⁻⁷ | <2.13 × 10⁻¹⁰ |

TABLE 5
MIC Values (mg/L) for EBO Selected Isolates of
M. avium ATCC 700898 Compared to the Parent Strain

| Drug | WT | 64-3A | 64-4A | 32-5A | 32-8A | 16-3A | 16-5A |
|---|---|---|---|---|---|---|---|
| EBO | 0.5 | >128 | >128 | >128 | >128 | >128 | 64 |
| AMK | 32 | 32 | 32 | 32 | 32 | 32 | 32 |
| BDQ | 0.06 | 0.12 | 0.06 | 0.03 | 0.12 | 0.06 | 0.06 |
| CLR | 2 | 0.5 | 2 | 1 | 0.5 | 0.5 | 1-0.5 |
| CFZ | 0.5 | 0.5 | 0.5 | 0.5 | 0.25 | 0.25 | 0.25 |
| EMB | 4 | 4 | 8 | 4 | 8 | 4 | 4 |
| RFB | 0.06 | 0.06 | 0.25-0.5 | 0.06 | 0.125 | 0.06 | 0.06 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of treating a non-tuberculosis Mycobacteria infection or a non-tuberculosis Mycobacteria-associated disease in a human in need thereof, comprising: administering epetraborole or a hydrate, solvate, or salt thereof, and ethambutol to the human,
thereby treating the non-tuberculosis Mycobacteria infection or the non-tuberculosis Mycobacteria-associated disease in the human.

2. The method of claim 1, further comprising administering a rifamycin, or a salt thereof, or rifamycin, or a salt thereof, to the human.

3. The method of claim 2, wherein the rifamycin is rifampicin (rifampin), rifabutin, rifapentine, or rifaximin.

4. The method of claim 1, further comprising administering a macrolide, or a salt thereof, to the human.

5. The method of claim 4, wherein the macrolide is clarithromycin or azithromycin.

6. The method of claim 1, wherein a pharmaceutically acceptable salt of epetraborole is administered to the human.

7. The method of claim 6, wherein the pharmaceutically acceptable salt of epetraborole is epetraborole hydrochloride.

8. The method of claim 1, wherein the non-tuberculosis Mycobacteria is rapidly growing.

9. (canceled)

10. The method of claim 1, wherein the non-tuberculosis Mycobacteria is slowly growing.

11. (canceled)

12. The method of claim 1, wherein the non-tuberculosis Mycobacteria is selected from the group consisting ofM. abscessus, M. avium complex (MAC), M. chelonae, M. fortuitum, M. gordonae, M. kansasii, M. mucogenicum, M. peregrinum, M. xenopi, M. intracellulare M. imarseillaise, M. timonense, M. bouchedurhonense, M. colombiense. M. vuilneris, and M. chimaera.

13. (canceled)

14. The method of claim 1, wherein the non-tuberculosis Mycobacteria is Mycobacterium avium complex.

15. (canceled)

16. (canceled)

17. (canceled)

18. (canceled)

19. (canceled)

20. The method of claim 1, wherein the human further has cystic fibrosis, chronic obstructive pulmonary disease, chronic thromboembolic pulmonary hypertension, an interstitial lung disease, post-inflarnatory lung fibrosi brohnchiectasis, a neoplastic disease. diabetes mellitus, bronchial asthma. hypothyreosis. mediastinal cyst, or rheumatoid arthritis.

21. (canceled)

22. The method of claim 20, wherein the interstitial lung disease is idiopathic pulmonary fibrosis, sarcoidosis, or proteinosis, and the neoplastic disease is myelofibrosis or lung cancer.

23. The method of claim 1, wherein the human previously suffered from tuberculosis.

24. The method of claim 1, wherein the infection is in the lung of the human.

25. The method of claim 1, wherein the infection is in two or more organs in the body.

26. The method of claim 1, wherein the infection is in the lymph nodes.

27. The method of claim 1, wherein the infection or the non-tuberculosis Mycobacteria-associated disease is treatment-naive.

28. The method of claim 1, wherein the infection or the non-tuberculosis Mycobacteria-associated disease is treatment-refractory.

29-34. (canceled)

35. The method of claim 1, wherein the non-tuberculosis Mycobacteria-associated disease is non-tuberculosis Mycobacteria-pulmonary disease, disseminated non-tuberculosis Mycobacteria disease, non-tuberculosis Mycobacteria-associated lymphadenitis, Mycobacterium avium complex (MAC) pulmonary disease, disseminated Mycobacterium avium complex (MAC) disease, and Mycobacterium avium complex (MAC)-associated lymphadenitis.

36. (canceled)

37. The method of claim 1, wherein the non-tuberculosis Mycobacteria-associated disease is nodular bronchiectasis or fibrocavitary.

38. (canceled)

39. (canceled)

40. (canceled)

41. The method of claim 1, wherein the non-tuberculosis is selected from the group consisting of M. abscessus, M. arabiense, M. aromaticivorans, M. bacteremicum, M. barrassiae, M. bourgelatii, M. celeriflavum, M. chelonae, M. crocinum, M. franklinii, M. fukienense, M. hippocampi, M. insubricum, M. iranicum, M. litorale, M. llatzerense, M. monacense, M. pallens, M. rufum, M. rutilum, M. salmoniphilum, M. sediminis, and Mycobacterium setense, M. algericum, M. alsiense, M. arosiense, M. bouchedurhonense, M. engbaekii, M. europaeum, M. fragae, M. heraklionense, M. indicus pranii, M. koreense, M. kumamotonense, M. kyorinense, M. lepromatosis, M. liflandii, M. longobardum, M. mantenii, M. marseillense, M. minnesotense, M. noviomagense, M. paraffinicum, M. paragordonae, M. parakoreense, M. paraseoulense, M. paraterrae, M. riyadhense, M. senuense, M. seoulense, M. sherrisii, M. shigaense, M. shinjukuense, M. simulans, M. sinense, M. stomatepiae, M. timonense, M. vulneris, and M. yongonense.
